(19) United States
(12) Patent Application Publication
(10) Pub. No.: US 2006/0203195 A1
(43) Pub. Date: Sep. 14, 2006

(54) Integrated ocular examination device

(76) Inventors: Bret C. Squire, Ann Arbor, MI (US); Bogdan Pathak, Lawrence, KS (US); Sandesh Malpure, Novi, MI (US)

Correspondence Address: GIFFORD, KRASS, GROH, SPRINKLE & CITKOWSKI, P.C, PO BOX 7021, TROY, MI, 48007-7021, US

(21) Appl. No.: 11/077,439

(22) Filed: Mar. 10, 2005

(51) Int. Cl. A61B 3/10

(52) U.S. Cl. 351/211

(57) ABSTRACT

An imaging device for use in ocular investigations and including a body incorporating a light creating projector for issuing a collimated light source. A digital micromirror device being positioned to intercept the collimated light source, the micromirror device reflecting the light source in a specified pattern and in at least one of first and second directions. A control system connected to the micromirror device and interfacing with at least one processor driven input/output device, the control system selectively reflecting the pattern in directions towards and away from a patient's eye.

### BACKGROUND OF THE INVENTION

1. Field of the Invention

The present invention relates generally to ocular examination and therapeutic devices. More specifically, the present invention teaches an adaptive collimated image device, incorporating the features of a collimated light source and digital micromirror device, and which combines the functional aspects of a number of ophthalmic tools into a single condensed enclosure digitally managed and interfaceable with hardware/software components. The collimated light waves are incident upon the digital micromirror device (DMD) at such an angle to each individual micromirror and to give rise to one of at least two reflected paths.

2. Description of the Prior Art

The prior art is well documented with various examples of digital imaging devices. Central to such applications is the digital micromirror device (DMD) which consists of a two-dimensional array of micromirrors on the order of a 16 μm (micrometer) square etched on a semiconductor chip. Each micromirror exhibits two symmetric pivot positions that are controlled individually through electrostatic forces. Upon illuminating a collimated light source into the array, the individual micromirrors together reflect multiple collimated beams of light into an organized array pattern of pixels to create a projected image.

Examples of DMD devices include the micromirror optical switch set forth in U.S. Pat. No. 6,618,520, issued to Tew, and which teaches an optical switch using an array of mirrors which selectively reflect light from an input fiber to either of a first or second output fiber. Each fiber is held in a ferrule which aligns the fiber with a focusing device, and which in turn causes the beam of light to either collimate, diverge, or converge.

The focusing device associated with each output fiber collects the beam of light for input into the output fibers. Light from the input fiber strikes a first mirror or group of mirrors in the array and is selectively deflected to a second mirror or group of mirrors associated with an output fiber by reflecting the beam of light from a retro-reflector between the fibers. The second mirror receives the beam from the retro-reflector and reflects it to the output fiber associated with the second mirror. Of note, the pivot mirrors in this design are not micromirrors and do not provide for electrostatic switching.

U.S. Pat. No. 6,453,083, issued to Husain et al., teaches a further number of micromachined optomechanical switching cells and matrix switches including such switching cells. One optomechanical switching cell includes a parallel plate actuator positioned on a substrate. A mirror is coupled to the actuator and is disposed to selectively redirect an incident optical beam.

An optomechanical matrix switch includes a substrate and a plurality of optomechanical switching cells coupled thereto. The matrix switch further includes an arrangement for monitoring the optical power incident upon, and output by, the matrix switch.

A still further example of an optomechanical matrix switch including collimator array is set forth in U.S. Pat. No. 6,445,841, issued to Gloeckner, and which teaches a substrate with a plurality of optomechanical switching cells coupled thereto. Each of the switching cells includes a mirror and an actuator. The matrix switch further includes an array of collimator elements, each being in optical alignment with one of the optomechanical switching cells.

Also disclosed is a distributed matrix switch including first and second optomechanical matrix switches. The first and second optomechanical matrix switches respectively include first and second pluralities of optomechanical switching cells mounted upon first and second substrates. A collimator array is interposed between the first and second matrix switches in optical alignment with the first and second pluralities of optomechanical switching cells.

A first example of an application including a DMD device is such as is disclosed in U.S. Patent Application Publication No. 2004/0051847, to Vilser, and which teaches a device and method for imaging, stimulation, measurement and therapy, in particular for the eye. A further example is set forth in WO 00/21432, to Verdooner et al., and which teaches an ocular fundus camera for digitally imaging an eye to be tested, an illuminating path for projecting an illuminating beam from the light source to the fundus, and an imaging path for viewing a desired portion of the fundus.

The light source in Verdooner is a halogen lamp and the illumination path includes a filter, collimating lens, mirror mask, and objective lens. Further, the objective lens is an aspheric lens and is preferably positioned about 25 mm from the cornea of the eye. The imaging path includes an objective lens, mask, and a relay lens. The fundus camera further includes a receiving member which is a CCD camera that converts the received light into a digital image and which can be simultaneously viewed and stored. The fundus camera is focused on the pupil to improve the depth of field, and the mask is positioned to block spurious light reflections which decrease the clarity of the digital images.

A final example drawn from the prior art is set forth in U.S. Pat. No. 6,246,504, issued to Hagelin et al., and which teaches a method of operating a micromechanical scanning apparatus including the steps of identifying a radius of curvature value for a micromechanical mirror and modifying a laser beam to compensate for the radius of curvature value. The identifying step includes the steps of measuring the far-field optical beam radius of a laser beam reflected from the micromechanical mirror, and in order to determine a focal-length value. The micromechanical scanning apparatus is operated by controlling the oscillatory motion of a first micromechanical mirror with a first micromechanical spring and regulating the oscillatory motion of a second micromechanical mirror with a second micromechanical spring.

### SUMMARY OF THE PRESENT INVENTION

The present invention teaches an adaptive collimated image device, incorporating the features of a collimated or selectively focused light source and a digital micromirror device (hereinafter DMD), which combines the functional aspects of a number of ophthalmic tools into a single condensed enclosure digitally managed and interfaceable with hardware/software components. The collimated light source is typically created by a focused bulb followed by a light integrator and collimating lens, the output of which is beamed onto the DMD device.

The collimated light waves are incident upon the digital micromirror device at such an angle to each individual micromirror to give rise to one of at least two reflected paths, these being reflected in directions towards and away from the patient's eye. A power source, either AC outlet or battery supplied, powers the device which may further include a processor driven control system which in turn interfaces with a PC and/or other suitable input controller device such as a joystick or keyboard.

Additional embodiments include the provision of one or more mirrors, selectively pivotable and operable to modify the perceived origin of the beam paths directed to the eye. The mirrors are typically placed subsequent to the micromirror array; however, they can, in certain instances, be positioned between the illumination source and the DMD.

A further variant includes the provision of a two-segment mirror, which causes portions of substantially collimated beam paths to extend toward the eye at slight angles relative to each other, this being to accomplish measurement of a desired visual acuity of the patient's eye by inducing selective accommodation of the eye's crystalline lens. A virtual test screen can be spaced at a given focal length from the patient's eye and upon which may be projected upper and lower overlapping images, the degree of overlap determining a given visual acuity and focal distance. In addition to visual acuity testing capabilities, the imaging device of the present invention can be utilized to diagnose other pathologies and provide ocular therapy, such as in the form of flicker photometry, in order to stimulate the user's eye, creating psychoperceptual responses that can be used for diagnostic purposes.

### BRIEF DESCRIPTION OF THE DRAWINGS

Reference will now be made to the attached drawings, when read in combination with the following detailed description, wherein like reference numerals refer to like parts throughout the several views, and in which:

FIG. 1A is a first representational illustration of a collimated light source projected upon a selected face of a digital micromirror device (hereinafter DMD) comprising a plurality of micromirrors according to the present invention;

FIG. 1B is a succeeding illustration of a first reflected angle path redirected in a vector normal to the DMD when the selected micromirror is in an active and illuminating position;

FIG. 2A is a first illustration of a path of a selected collimated light beam being redirected by an associated micromirror arranged in a first angular position corresponding to the micromirror in an “ON” position and which is non-parallel to a normal vector extending from a face of the DMD and whose redirection is parallel to the normal vector extending from the face of the DMD;

FIG. 2B is a second path illustration of a selected collimated light beam and by which the beam is redirected by an associated micromirror in a direction substantially corresponding to that illustrated in FIG. 2A;

FIG. 2C is a third path illustration of a selected collimated light beam and a selected light beam reflected from the target by which the beams are simultaneously redirected to two separate locations by an associated micromirror arranged in a second angular position corresponding to the micromirror in an “OFF” position (FIG. 2D) and whose redirected paths are non-parallel to a normal vector extending from a face of the DMD;

FIG. 3 is an illustration of an embedded control system interfacing between the DMD device and at least one of a processing device or input device including such as a joystick and/or keyboard;

FIG. 4 is a modified illustration to the system in FIG. 3 and illustrating a power source for operating the processing device, DMD and collimated light source;

FIG. 5 is an illustration of the first preferred embodiment of the ocular examination device, by which the components illustrated in FIGS. 1-4 are shown together;

FIG. 6A is an illustration of a visual accommodation cue test in which a collimated path is reflected from a DMD upon a two segment mirror, which in turn causes portions of the collimated paths to extend toward the eye at a slight angle, forming a virtual image path, extending rearward from the mirror, overlapping upon a virtual test screen and which divides the DMD image into upper and lower halves;

FIG. 6B is a succeeding illustration to that shown in FIG. 6A and which shows the virtual test screen and split DMD images corresponding to a visual acuity test;

FIG. 7A is an illustration of an adaptive collimated image, such as according to the illustration of FIG. 5, modified to give a visual accommodative cue through the use of a synchronized pivotable mirror;

FIG. 7B is an alternate illustration to FIG. 7A, showing the synchronized pivotable mirror placed between the path of projection from a collimated light source and the DMD;

FIG. 8A is an illustration of a modification of FIG. 7A and by which a second pivotable mirror is arranged such that it controls an orthogonal axis of rotation of a collimated image path compared to the first pivotable mirror's axis of rotation and directed in plural fashion towards a patient's eye;

FIG. 8B is ninety degree rotated view of FIG. 8A, from the perspective of the observer's eye, and by which a collimated image path is shown reflected from the DMD and towards the second pivotable mirror;

FIG. 9A is an illustration of a modification of FIG. 7B and by which a second pivotable mirror placed to control an orthogonal axis of rotation of the path of projection from a collimated light source, and compared to the first pivotable mirror's axis of rotation of the path of projection from the collimated light source;

FIG. 9B is a ninety degree rotated view of FIG. 9A, from the perspective of the observer's eye and showing the arrangement of mirrors for redirecting the collimated light path to the DMD;

FIG. 10A is an illustration of a modification of the ocular examination device of FIG. 5 and by which a gimbaled mirror is placed between the collimated image path reflecting off the DMD and a patient's eye;

FIG. 10B is a ninety degree rotated view of the modification of FIG. 10A, from the perspective of the observer's eye;

FIG. 11A is an illustration of a modification of the ocular examination device of FIG. 5 and by which a target screen is placed between the collimated image path reflecting off the DMD and a patient's eye;

FIG. 11B is a slightly rotated view of the modification of FIG. 11A;

FIG. 12A is an illustration of a modification of the ocular examination device of FIG. 11A and FIG. 11B and by which a refractive lens system is placed between the collimated image path reflecting off the DMD and the target screen; and

FIG. 12B is a slightly rotated view of the modification of FIG. 12A.

### DETAILED DESCRIPTION OF THE PREFERRED EMBODIMENTS

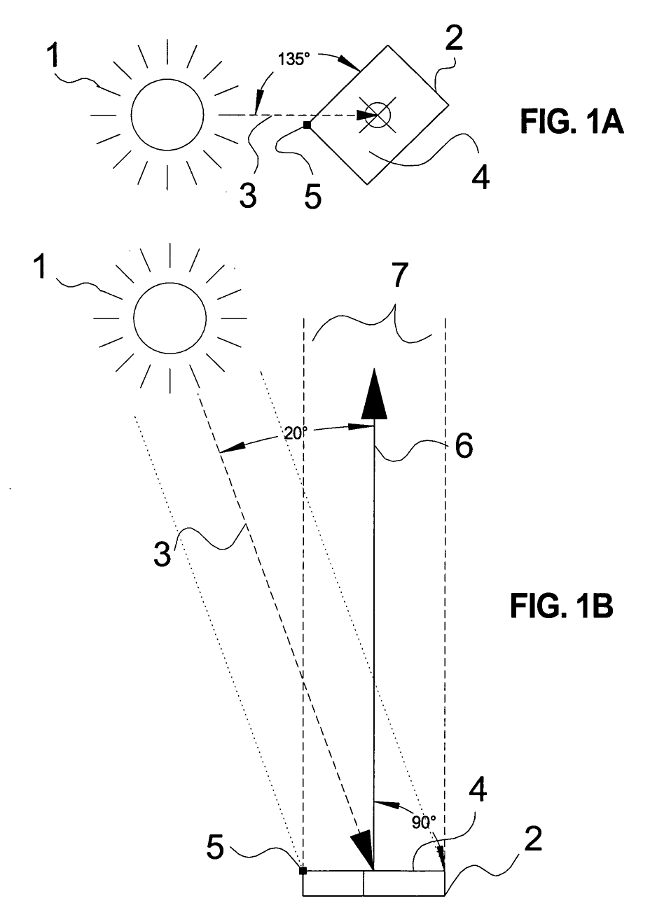

Referring now to FIG. 1A, a first representational illustration is shown of a collimated light source 3 projected upon a selected face 4 of a digital micromirror device 2 (hereinafter DMD), such typically including a plurality of micromirrors individually formed on the face 4, according to a first preferred embodiment of the present invention with the face 4 of the DMD 2 pointed out of the page. As previously explained, the adaptive collimated image device incorporates the features of a collimated light source and digital micromirror device, in order to combine the functional aspects of a number of ophthalmic tools into a single condensed enclosure digitally managed and interfaceable with hardware/software components.

A light or illuminating source is generally referenced at 1 and, in a preferred embodiment, may be constructed of components similar to those used in a digital light processing (or DLP) projector. Although not shown, such components may include a bulb with a focusing housing followed by a condensing lens, an aperture at the focal point and a second condensing lens that collimates the output from the aperture which is incident onto the DMD 2. In order to create a uniform illumination intensity the aperture can be replaced with a light integrator rod. To add color, a color wheel containing color filter segments can be placed after the light integrator rod or before the aperture if the integrator rod is not used. To prevent harm to the eye, neutral density, UV and IR filters can be used. The modifications and additions to the illuminating source components will depend on the spectral output of the bulb, the perceptual response of the eye, and the limits of safety for the eye. The present invention contemplates the creation of a plurality of parallel, or collimated, light beams by any mechanism available, and which are illustrated in a path of projection 3.

The DMD 2 is constructed as substantially previously described and again includes a two-dimensional array of micromirror squares etched on a semiconductor chip and further referenced by face 4 associated with the DMD chip. The DMD further includes a manufacturer marking 5 and which, as specified upon a manufacturer's technical sheet, determines the positioning of the DMD at a specified angle relative to a normal vector extending from its face 4 (as further referenced at 6 in FIG. 1B).

Each micromirror further exhibits two or more symmetric pivot positions that are controlled individually and such as through electrostatic forces. Upon illuminating a focused, or collimated, light source incident onto the array, the individual micromirrors together reflect collimated beams of light into an organized pattern of pixels to create a projected image. In practice, each micromirror produces a time varying bundle of light which corresponds to an element on the overall beam front (or BEFEL, which designates a beam front element).

It is further envisioned that the light emitted should encompass a significant area of the active portion of the DMD 2 and exhibit a uniform intensity. Referring again to FIG. 1B, the collimated light source 1 need further be placed far enough away from the DMD 2 so as not to obstruct a first reflected path 7 of collimated light beams and which is not parallel relative to the normal vector 6 extending from the DMD face 4. In the example given in FIG. 1B, a 20 degree angular offset is referenced between the collimated path of projection 3 and the reflected path 7. The actual value of the angular offset will depend on the manufacturer's specifications for the DMD.

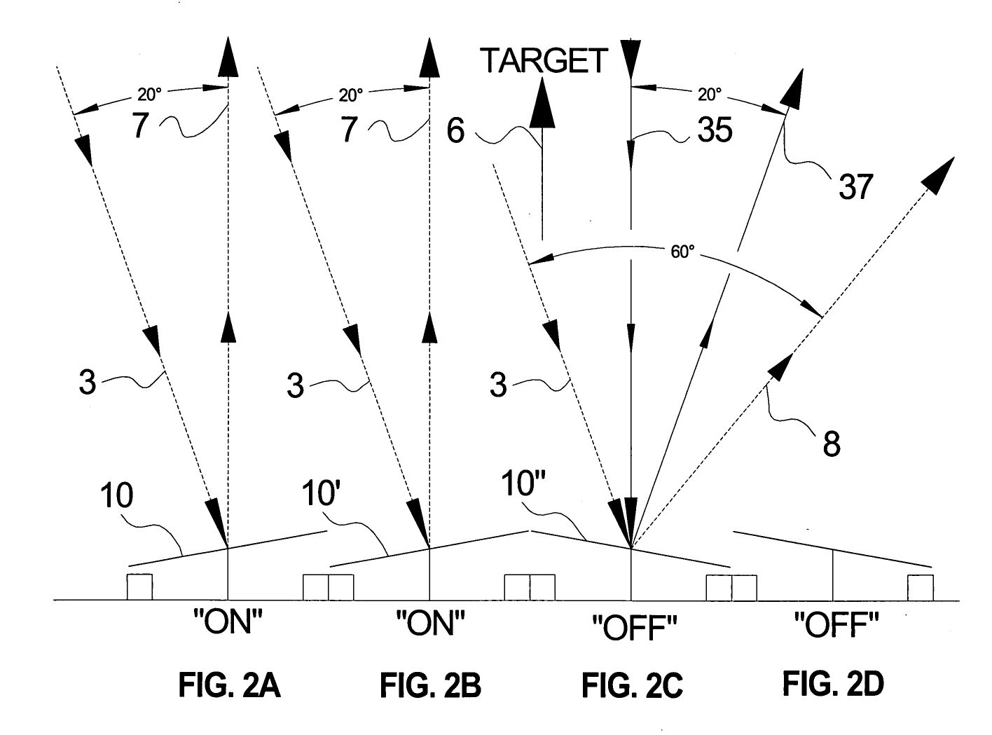

Referring now to FIG. 2A, a first illustration of a path of a selected collimated light beam is illustrated as being redirected by an associated micromirror 10 (forming a portion of a DMD. In particular, the micromirror 10 is arranged in a first angular position corresponding to the micromirror being in an “ON” position and which is non-parallel to a normal vector 6 (such as previously illustrated at 6 in FIG. 1B) extending from the illuminated face 4 of the DMD 2. The collimated light path is again referenced at 3 and a reflected path 7 extends parallel with the normal vector 6. Additionally, the first reflected path 7 should still exhibit a relatively collimated (parallel) nature and should have a uniform intensity when all the micromirrors 10 are in a constant “ON” position.

Referring now to FIG. 2B, a second illustration is shown of a selected collimated light beam 3 being redirected by an associated and angled micromirror 10′ in a manner substantially corresponding to that illustrated in FIG. 2A. FIGS. 2A and 2B give an example of the collimated, or parallel, nature of the projected image from the DMD.

FIG. 2C is a third path illustration of a selected collimated light beam 3 and by which the beam is redirected at 8 by an associated micromirror arranged in a second angular position corresponding to the micromirror in an “OFF” position, and which is non-parallel to a normal vector 6 extending from the face of the DMD 4, by further reference to a direction 8 of the reflected beam path relative to the angled micromirror 10”. FIG. 2C also demonstrates a simultaneous fourth illustration of a selected light beam 35 originating from the target and by which the beam is redirected at 37 by an associated micromirror arranged in a second angular position corresponding to the micromirror in an “OFF” position (see FIG. 2D), and which is non-parallel to a normal vector 6 extending from the face 4 of the DMD 2, by further reference to the direction 37 of the reflected beam path relative to the angled micromirror 10″. The redirected beam 37 from the target is non-parallel to the redirected beam 8 from the illuminating light source giving the ability to image the eye via an ocular scope or other imaging component without interference from the light beams 3 originating from the collimated light source. The view of FIGS. 2A-2C are intended to illustrate and exemplify the ability of the present invention to provide for iterative imaging to and from the eye, and such as is associated with various device driven ocular procedures known in the art. It is also understood that the angular offsets of the light beams 3, 7, 8, 35 and 37 can be adjusted according to desired manufacturing specifications. For example, suggested angles of 20 degrees are illustrated for incident beams 3 and 35, however it is understood that such angles may easily vary within the scope of the invention.

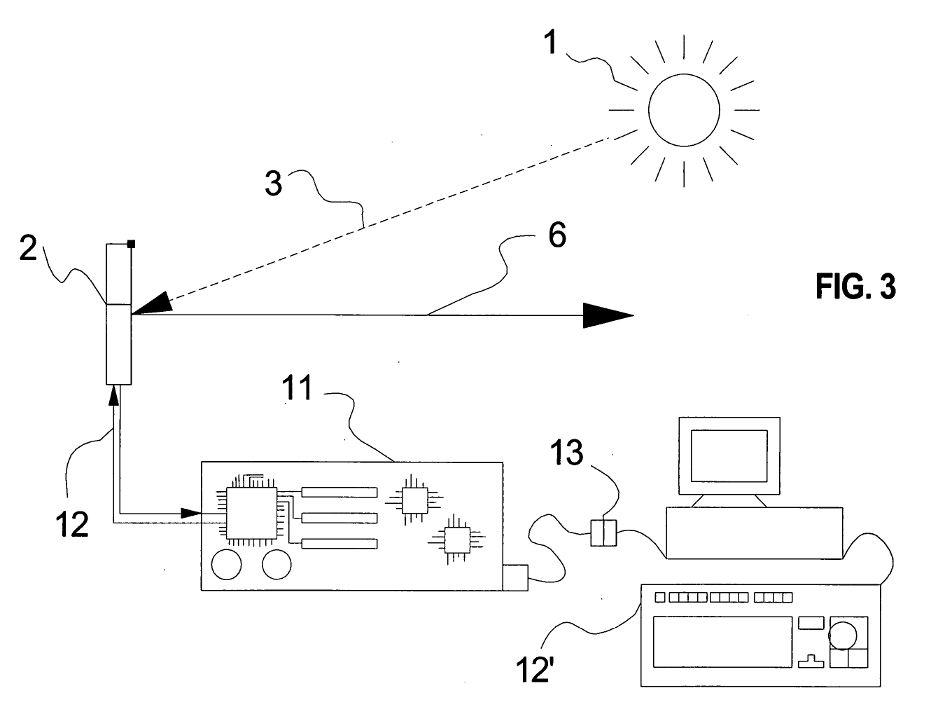

Referencing now FIG. 3, an illustration is shown of an embedded control system 11 interfacing, see at 13, between the DMD 2, see at 12, and at least one of a processing device, computer or other input device 12′ including such as a joystick and/or keyboard 12′. The specifications of the interface 12 between the DMD 2 and the embedded control system 11 are determined by the DMD manufacturer. The interface 13 is further understood to include a communication port extending to the computer/input device and the embedded control system 11 can exist as a fully integrated computer system with enough memory, input devices, and output devices as are necessary. As is commercially known, Texas Instruments Corporation produces a development board that can control such devices.

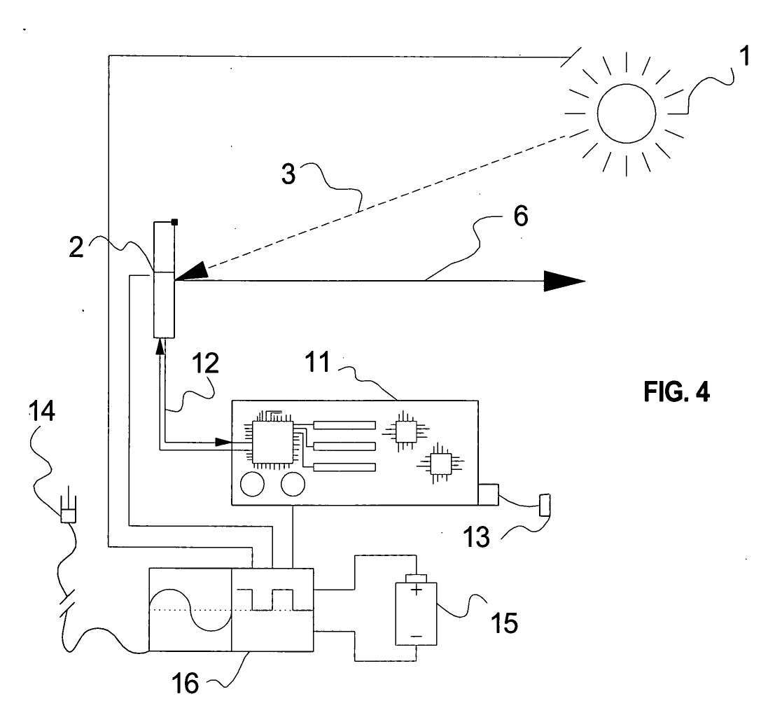

Referring to FIG. 4, a modified illustration to the system in FIG. 3 is shown and which illustrates a converter 16 for modifying a standard electrical power source operating the processing device 11, DMD 2 and collimated light source 1. Specifically, the converter 16 operates to convert an electrical wall outlet source 14 (such as an AC outlet power) into the specified power source requirements of the DMD 2, embedded system 11 and the collimated light source 1. Alternately, the converter 16 can convert a battery 15 source into the specific power requirements of the previously stated system components. It is further understood that the collimated light source can contain extra components such as a color wheel motor, commonly found in the DLP projector, that may also require a power input, but for simplicity is again generally referenced as the collimated light source 1 in FIG. 4 to represent any variations known in the art.

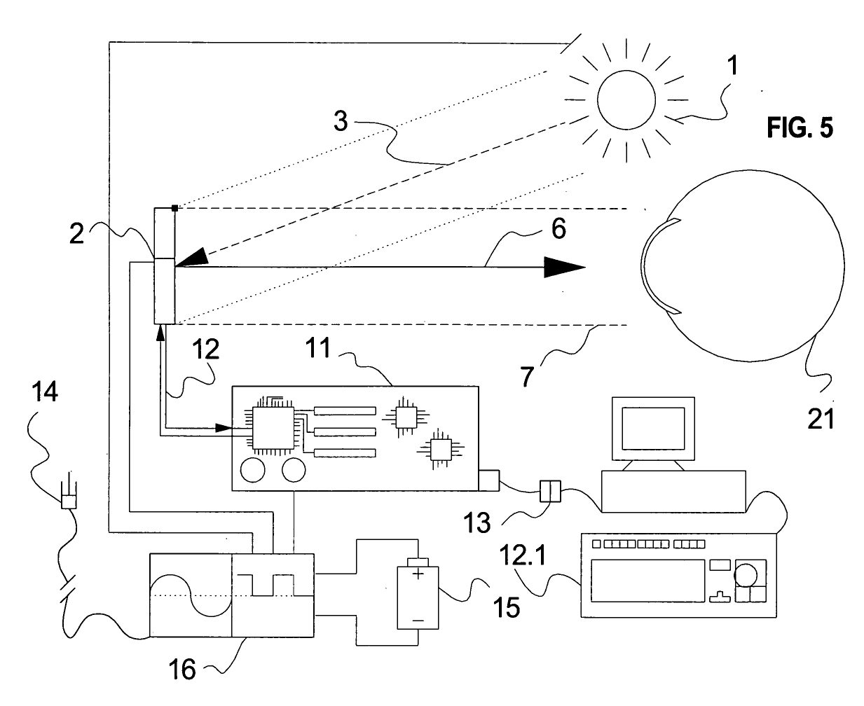

FIG. 5 is a further modified illustration of the arrangements of FIG. 3 and FIG. 4, and by which the first preferred embodiment of the ocular examination device can give a visual accommodative cue where the stimulus image (affecting a virtual focal distance) can be changed through programming. Specifically, a beam path 7 reflected from the DMD 2 is directed towards a patient's eye 21, such as within a range corresponding to the normal vector, and in a manner consistent with the ON/OFF positions of FIGS. 2A-2D.

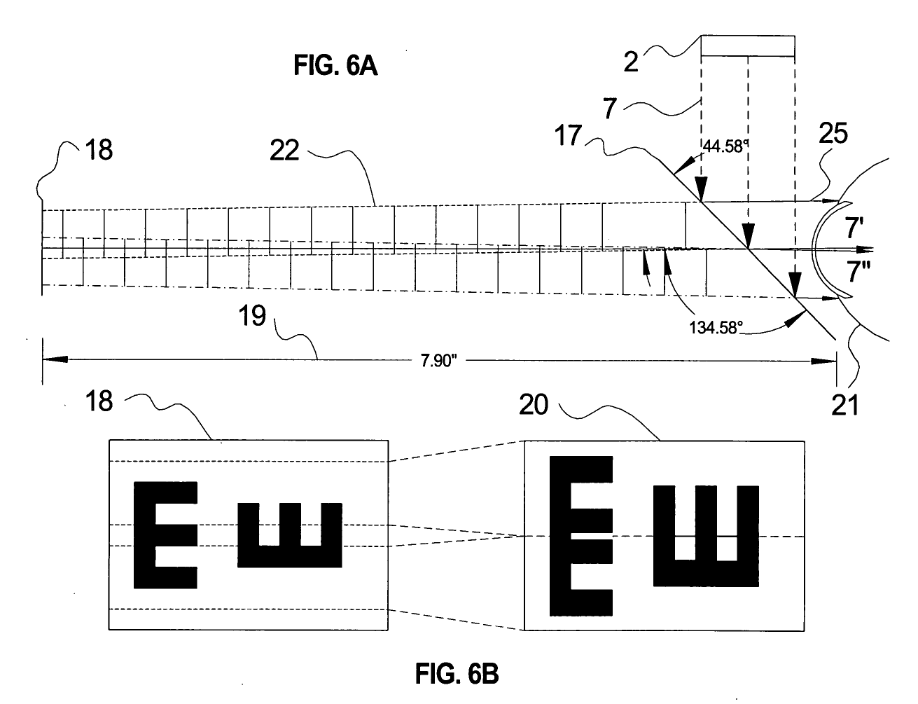

The ocular examination device of FIG. 5 can be modified to give a stronger visual accommodative cue where a virtual focal distance can be changed through programming and a special segmented mirror. FIG. 6A is an illustration of a visual accommodation test, and in which a collimated path is reflected from the DMD 2 upon a two-segment mirror 17. This in turn causes portions 7′ and 7″ of the collimated paths to extend toward the eye 21 at a slight angle relative each other.

A virtual image path 22, extending rearward from the mirror 17, overlaps upon a virtual test screen 18 which divides the DMD image into upper and lower halves. Specifically, and referencing FIG. 6B, the virtual test screen 18 exhibits split DMD images 20 corresponding to a visual acuity test. The top half covers the upper overlapping image on the virtual test screen 18 and the bottom half the lower part of the overlapping image. The more overlap which exists between the top and bottom halves of the DMD image, the smaller a virtual focal distance 19 and the closer the virtual test screen 18 is to the eye. Ideally, the angle of the mirror segments 17 should be determined by the farthest virtual focal distance necessary, such for visual acuity testing being set about 20 feet.

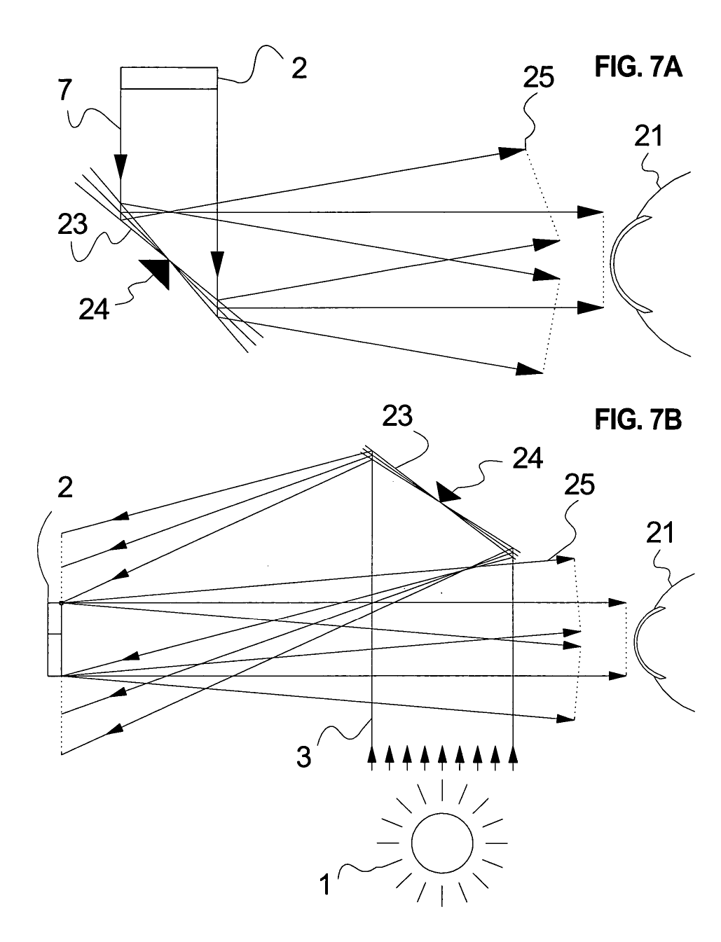

The ocular examination device of FIG. 5 can be modified to give a stronger visual accommodative cue where a virtual focal distance can be changed through programming and a synchronized pivotable mirror. Referencing now FIG. 7A and FIG. 7B, illustrations showing a collimated image, such as according to the illustration of FIG. 5, are modified to give a stronger visual accommodative cue through the use of a synchronized mirror 23 arranged about a pivot 24. Similar to the segmented mirror of FIG. 6A, the synchronized mirror can change the perceived origin of the virtual image path via its pivot position. Changing the image displayed on the DMD 2 and the pivot position of the mirror over time, to reflect the desired origin of the virtual image path, will give the eye an accommodative cue. The pivotable mirror 23 can either be placed between the collimated image path 7 reflecting off of the DMD 2 and the eye 21, or between the path of projection 3 from the collimated light source 1 and the DMD 2, as shown respectively in FIGS. 7A and 7B. It is further understood that the synchronized mirror 23 includes a motorized actuator to control the mirror's pivot position and is powered and controlled by the system illustrated in FIG. 5.

FIG. 7B is an alternate illustration to FIG. 7A and shows a pair of angled collimated image paths 25, reflected from the DMD 2, and such that the paths are directed towards the eye 21, such as which can be associated, without limitation, with a patient in a diagnostic application, as well as any user or observer in both diagnostic as well as non-diagnostic applications, in a time based and multiple fashion in order to provide a stronger visual accommodative cue. What results, from FIG. 7A or FIG. 7B, is something similar to the mirror segments 17 of FIG. 6, only instead of the collimated image path 7 being divided into two or more angular based collimated paths 25, the entire collimated image path has a time based angular direction. This allows for more image resolution and area of coverage for the particular angular based collimated image paths 25.

Depending further upon the angular resolution of the pivotable mirror 23, a multitude of angular based collimated image paths 25 can be produced, allowing for more precise placement of the virtual image paths 22 that overlap the test screen 18 (see again FIG. 6A). Accordingly, and the more angular based collimated image paths 25 that can be directed towards the eye 21, the stronger the visual accommodative cue becomes. When the eye is not focused on the desired virtual test screen the image will appear out of focus.

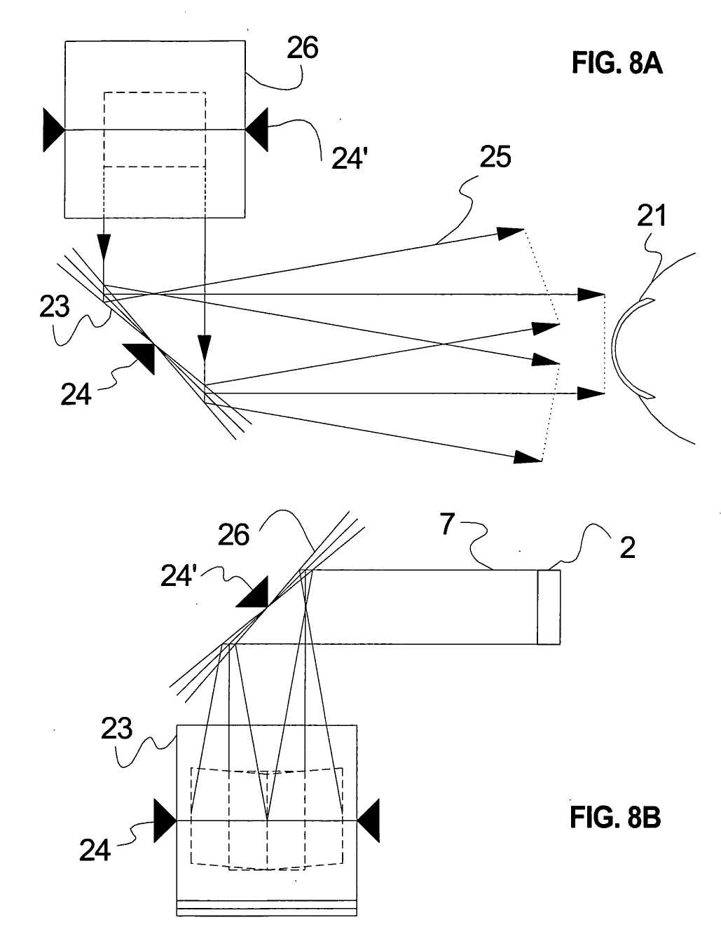

The ocular examination device of FIG. 5, with modifications from FIG. 7B, can be further modified to give a stronger visual accommodative cue where a virtual focal distance can be changed through programming and two synchronized pivotable mirrors. Referencing further FIG. 8A, a modification of FIG. 7B is provided by which a second synchronized mirror 26 is pivotally 24′ arranged, such that it controls an orthogonal axis of rotation of collimated image paths 25 compared to the first pivotable mirror's 23 axis of rotation 24, and directed in plural and time-varying fashion towards a patient's eye 21. FIG. 8B is ninety degree rotated view of FIG. 8A and by which the collimated image path 7 is shown reflected from the DMD 2 and towards the second pivotable mirror 26, and which is reflected off of the first pivotable mirror 23 and towards the patient's or other user's/observer's eye 21, which is looking into the page.

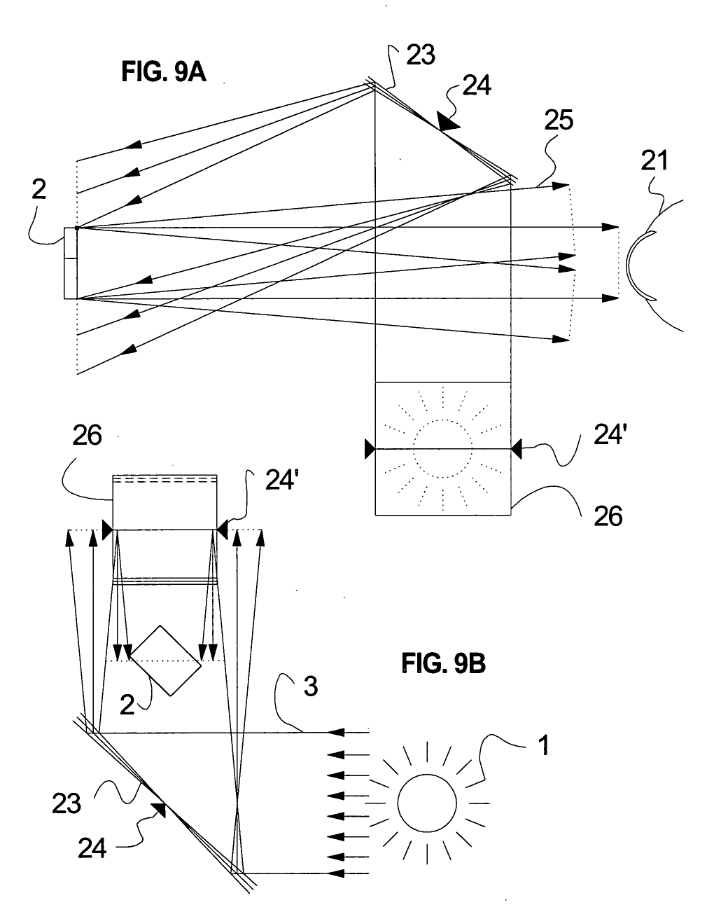

The ocular examination device of FIG. 5, with modifications from FIG. 7B, can be further modified to give a stronger visual accommodative cue where a virtual focal distance can be changed through programming and two synchronized pivotable mirrors placed between the DMD 2 and the collimated light source 1. Referring to FIG. 9A, an illustration is shown of a second synchronized mirror 26 placed to control an orthogonal axis of rotation 24′ of the collimated path of projection 3 from the collimated light source 1, and compared to a first pivotable mirror's 23 axis of rotation 24 of the collimated path of projection 3 from the collimated light source 1 which is shining into the page. Specifically, both mirrors 23 and 26 operate off of their respective pivots 24 and 24′ in order to create a beam path 25 of multiple rays directed to the eye 21. FIG. 9B is a ninety degree rotated view of FIG. 9A and shows the arrangement of mirrors for redirecting the collimated path of projection 3 of light to the DMD 2.

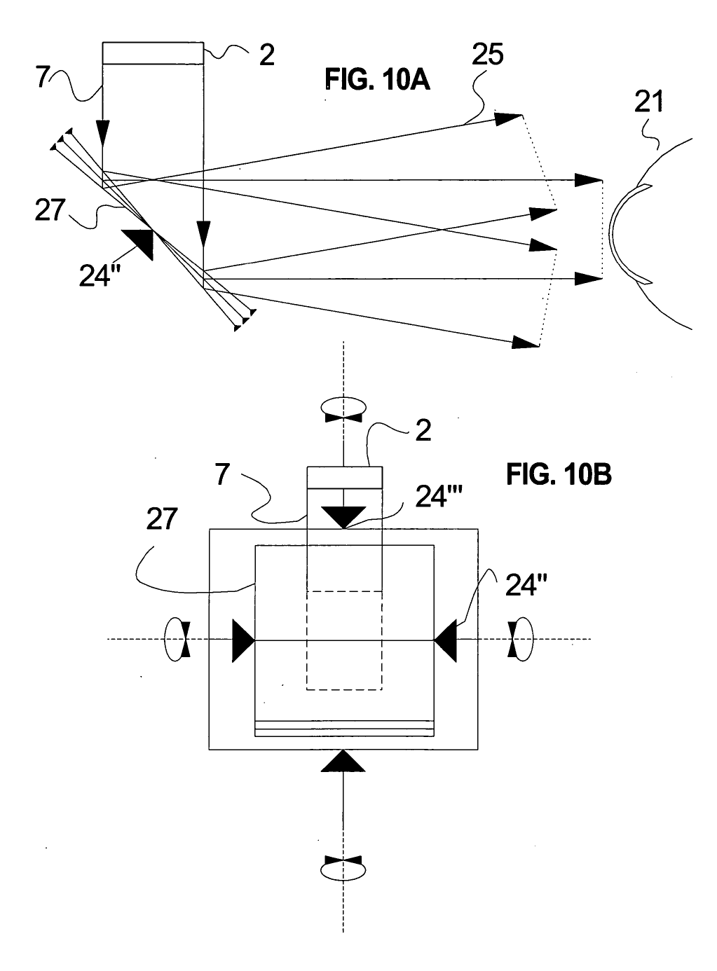

Finally, the ocular examination device of FIG. 5 can be modified to give a stronger visual accommodative cue where a virtual focal distance can be changed through programming and a synchronized gimbaled mirror. Referring to FIG. 10A, an illustration is shown of a modification of the ocular examination device of FIG. 5, and by which a gimbaled mirror 27 (see pivots 24″ and 24′″) is placed between the collimated image path 7 reflecting off the DMD 2 and the patient's eye 21. FIG. 10B is a ninety degree rotated view of the modification of FIG. 10A which illustrates the multi-pivotal nature of the mirror 27, with the patient's eye 21 looking into the page. It is further understood that the synchronized gimbaled mirror 27 (see FIGS. 10A and 10B) includes one or more motorized actuators to control the mirror's pivot positions and is powered and controlled by the system illustrated in FIG. 5.

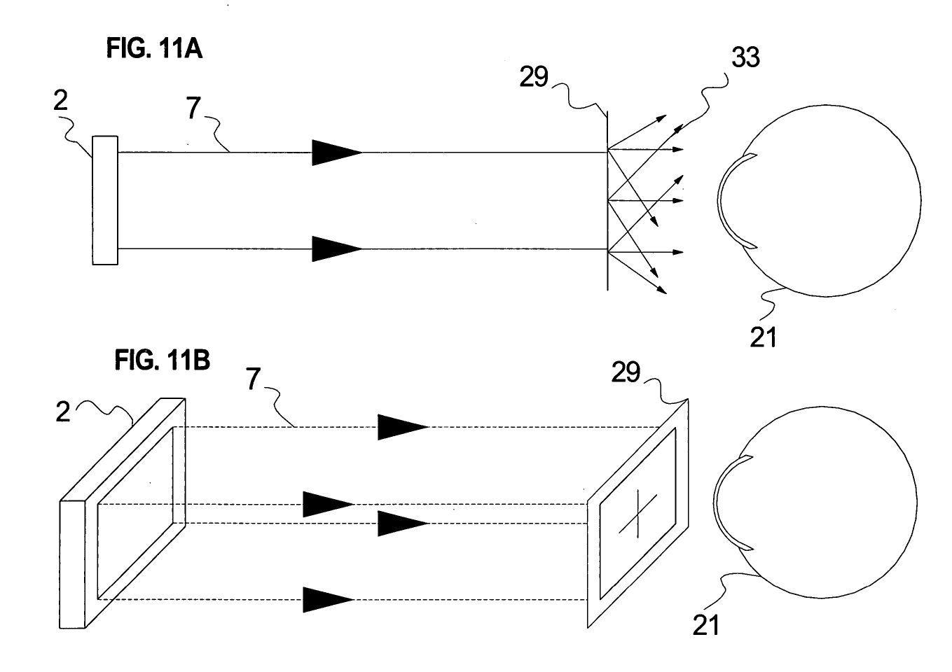

Referring to FIGS. 11A and 11B, the ocular examination device of FIG. 5 can be modified to create a dynamically controlled flicker photometer or other stimulus where a physical target screen 29 can be placed a specific distance from the eye 21 and the test image (displayed on screen 29) is controlled though software programming. FIG. 11A is an illustration of a flicker photometry test, and in which a collimated image path 7 is reflected from the DMD 2 upon a target screen 29. This in turn causes the collimated image path 7 to diverge from the target screen 29 into a scattered image path 33 towards the eye 21. This scattering can be the result of transmitting a collimated image path through a transmissive projection screen surface or reflected off a reflective projection screen.

Ideally, the target screen 29 would provide a nearly Lambertian surface or uniformly scatter each light ray path. The scattered image path 33 would allow the eye 21 to accommodate or focus onto the target screen 29. Those skilled in art of flicker photometry can establish the required specifications of the image on the target screen 29. Through programming these required specifications can be controlled dynamically. FIG. 11B is a slightly angled exploded view of the illustration in FIG. 11A.

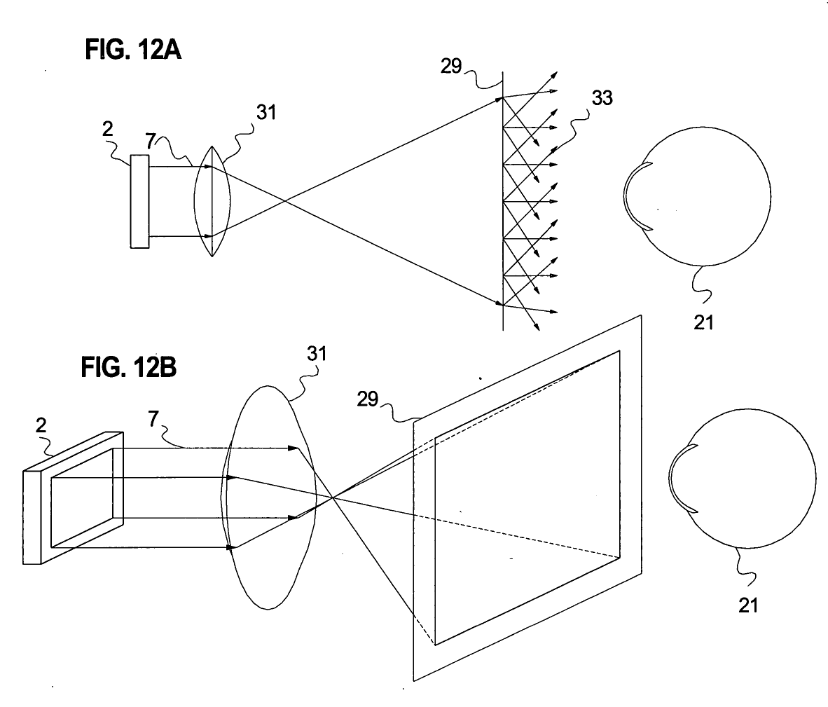

The ocular examination device of FIG. 11A can be modified to enlarge the area of the scattered image path 33 where a refractory lens system 31 is placed between the DMD 2 and the target screen 29. FIG. 12A is an illustration of a flicker photometry test, and in which a collimated image path 7 is reflected from the DMD 2 through the refractory lens system 31 and upon a target screen 29. This in turn causes the collimated image path 7 to diverge from the target screen 29 into a scattered image path 33, with a larger area than before, towards the eye 21. FIG. 12B is a slightly angled exploded view of the illustration in FIG. 12A.

Accordingly, the adaptive collimated image device functions as a virtual fixation point or virtual target generator which is useful for varying types of ocular examinations, including detection of abnormal states through subjective refraction, distant chart projection, and near chart projection. The collimated image device is the functional replacement of the skiascope, slit lamp, retinal camera, scanning laser ophthalmoscope, and flicker photometer. Additional therapeutic applications made possible by image device include its use as a novel and dynamic stimulus for more modern tests such as flicker photometry.

Having described our invention, other and additional preferred embodiments will become apparent to those skilled in the art to which it pertains, without deviating from the scope of the appended claims.

We claim:

1. An imaging device for use in ocular investigations, comprising:
a collimated light source;
a digital micromirror device positioned to receive said collimated light source, said micromirror device reflecting said light source in a specified pattern and in at least one of first and second directions; and
a control system connected to said micromirror device and interfacing with at least one of a processor and an input/controller device, said control system selectively controlling a reflection of said pattern in a direction towards a patient's eye.

2. The imaging device as described in claim 1, said collimated light source further comprising a light generating mechanism for producing a plurality of substantially parallel light rays.

3. The imaging device as described in claim 1, said digital micromirror device further comprising a plurality of micromirrors etched on a semiconductor chip, said micromirrors each being symmetrically pivoted through at least two positions and by electrostatic forces.

4. The imaging device as described in claim 1, said digital micromirror device reflecting a beam pattern corresponding to a selected one of a plurality of positions.

5. The imaging device as described in claim 4, each of said beam patterns corresponding to an angular offset relative to a vector extending normal to a face of said digital micromirror device.

6. The imaging device as described in claim 1, further comprising a power source in operable communication with at least one of said control system, digital micromirror device, and collimated light source.

7. The imaging device as described in claim 6, said power source further comprising an AC outlet supply.

8. The imaging device as described in claim 6, said power source further comprising a battery.

9. The imaging device as described in claim 1, further comprising said collimated path being reflected from said digital micromirror device upon a two-segment mirror, said mirror causing portions of said collimated paths to extend toward the eye at a slight angle relative each other.

10. The imaging device as described in claim 9, further comprising a virtual image path extending rearward from said mirror being exhibited on a virtual test screen separated by a given focal length from the eye, said screen exhibiting a pair of images corresponding to a visual acuity test, an upper half of said screen exhibiting an upper overlapping image and a bottom half exhibiting a lower overlapping image.

11. The imaging device as described in claim 1, further comprising an adaptive collimated image modified to give a visual accommodative cue through the use of a synchronized mirror arranged about a pivot, said mirror being placed between a substantially collimated image path reflecting off of said digital micromirror device and the eye.

12. The imaging device as described in claim 1, further comprising an adaptive collimated image modified to give a visual accommodative cue through the use of a synchronized mirror arranged about a pivot, said mirror being placed between said path of projection from said collimated light source and said digital micromirror device.

13. The imaging device as described in claim 1, further comprising a pair of angled collimated image paths reflected from said digital micromirror device and such that said paths are directed towards the eye in a time based and multiple fashion in order to provide a perception of multiple simultaneous visual accommodative cues.

14. The imaging device as described in claim 11, further comprising a second mirror being pivotally arranged such that it controls an orthogonal axis of rotation of a substantially collimated image path compared to said first pivotable mirror's axis of rotation, and such that said reflected pattern can be directed in plural fashion towards the eye.

15. The imaging device as described in claim 14, further comprising said first and second mirrors both operating off of a pivot in order to modify a beam path comprised of multiple rays directed to the eye.

16. The imaging device as described in claim 12, further comprising a second mirror being pivotally arranged such that it controls an orthogonal axis of rotation of a substantially collimated light path compared to said first pivotable mirror's axis of rotation, and such that said reflected pattern can be directed in plural fashion towards the eye via reflection off of the DMD.

17. The imaging device as described in claim 1, further comprising a gimbaled mirror placed between a substantially collimated image path reflecting off said digital micromirror and the eye.

18. An imaging device for use in ocular investigations, comprising:
a body incorporating a projector for creating a collimated light source;
a digital micromirror device positioned to intercept said collimated light source issued by said projector, said digital micromirror device reflecting said light source in a specified pattern and in at least one of first and second directions; and
a control system connected to said digital micromirror device and interfacing with at least one processor driven input/output device, said control system selectively reflecting said pattern in directions towards and away from a patient's eye.

19. The imaging device as described in claim 18, a power source in operative communication with at least one of said control system, digital micromirror device, and collimated light source.

20. The imaging device as described in claim 1, further comprising a target screen placed between a substantially collimated image path reflecting off said micromirror device and the patient's eye.

21. The imaging device as described in claim 20, further comprising a refractive lens system providing a means to enlarge the area of said collimated image path directed towards the target screen.
